(19) United States
(12) Patent Application Publication
(10) Pub. No.: US 2016/0184312 A1
(43) Pub. Date: Jun. 30, 2016

(54) TRANSDERMAL ADMINISTRATION SYSTEM FOR USE IN THE TREATMENT OF CHRONIC BRONCHOPULMONARY DISEASES

(71) Applicant: ABC FARMACEUTICI S.P.A., Torino (IT)

(72) Inventors: ALBERTO GIRAUDI, CINZANO, TORINO (IT); GIOVANNI GIRAUDI, TORINO (IT)

(21) Appl. No.: 14/908,701

(22) PCT Filed: Aug. 1, 2014
(86) PCT No.: PCT/IB2014/063610

§ 371 Date: Jan. 29, 2016

(30) Foreign Application Priority Data
Aug. 2, 2013 (IT) TO2013A000663

(51) Int. Cl. A61K 31/522; A61M 37/00; A61K 9/70

(52) U.S. Cl. CPC A61K 31/522; A61K 9/7046; A61M 37/00; A61K 9/7084; A61K 2121/00; A61M 2037/0007

(57) ABSTRACT

Transdermal administration system comprising doxofylline as the active ingredient, for use in the treatment of bronchopulmonary diseases having a broncho-obstructive component.

This invention relates to a transdermal administration system for use in treatment of diseases of the respiratory tract, particularly chronic bronchopulmonary diseases having a broncho-obstructive component, which is characterised by the use of Doxofylline as the active ingredient.

The invention also relates to Doxofylline in the context of its use in transdermal administration for therapeutic treatment of the abovementioned diseases.

Doxofylline, 2-(7′-theophylline methyl)-1,3-dioxolane is a xanthine derivative. The xanthines are a group of alkaloids commonly used for their effects as mild stimulants and bronchodilators, particularly in treatment of the symptoms of asthma. Numerous drugs, including acefylline piperazine, ambufylline, aminophylline, bamifylline, choline theophyllinate, dyphylline, enprofylline, etamifylline, etophylline, guatilline, proxyphylline, theobromine, theophylline and doxofylline belong to the xanthines family.

Xanthine derivatives are mild stimulants of the central nervous system and also stimulate the respiratory centres. Because of their wide spectrum of effects their therapeutic index is somewhat limited; their serum therapeutic level is generally of the order of 10-20 mg/ml and toxicity symptoms include tremor, nausea, nervousness and tachycardia/arrhythmia.

Various modulated release systems, including transdermal release systems which potentially offer a valuable solution, particularly in terms of compliance on the part of patients and in terms of efficacy of therapeutic response, have also been developed and proposed for xanthines in recent decades.

Despite the considerable interest in the transdermal administration route, the application of this administration route to a wider range of drugs has naturally been limited because of the significant barrier to penetration across the skin, which is primarily associated with the outer corneal layer of the epidermis. In general the daily dose of drug which can be administered by, for example, a transdermal patch is somewhat limited (e.g. 5-10 mg), limiting this method of administration to the most potent drugs.

Since the 90s many scientific studies have examined the percutaneous absorption characteristics of xanthine derivatives in human skin, using theophylline as the reference drug, in an attempt to obtain useful information for the development of transdermal administration systems.

The publication by Ademola J. I. et al. in Journal of Investigative Dermatology (1992), 98(3), 310-14 concludes that a high level of absorption potentiation is required for transdermal theophylline preparations to be able to produce therapeutic concentrations in plasma. The research was therefore aimed at identifying agents potentiating transdermal permeation. The publication by Thakur R. A. in Drug. Dev. Ind. Pharm. 2007, May; 33(5):513-21 examines the in vitro permeation of caffeine, theophylline and theobromine through the human skin with the aid of various chemical potentiating agents, including oleic acid. The administered flow of theobromine and theophylline was nevertheless still insufficient.

This invention is based on discovery of the fact that in a wholly unexpected way doxofylline has higher transepithelial permeability characteristics, which, in the context of the xanthines make it the ideal active ingredient for the implementation of transdermal administration systems useful in the therapeutic treatment of diseases of the respiratory tract, particularly in adults.

In particular the transdermal administration system which is the subject matter of the invention, containing doxofylline as the active ingredient, brings together the advantages in terms of compliance on the part of the patient and efficacy of therapeutic response which are typical of this form of administration, together with the known therapeutic benefits of doxofylline when administered systemically.

In particular it is known that doxofylline has high bronchodilatory activity with rare collateral effects, even in patients with associated morbidities and receiving multiple therapy. The safety data for the active ingredient and the efficacy of chronic treatment of chronic bronchopulmonary diseases having a broncho-obstructive component render doxofylline an extremely useful drug in long term treatment. In addition to this, long term treatment requires high compliance on the part of the patient, compliance which is well known to be improved through reducing administrations and by time-modulated administration systems. The transdermal administration route for doxofylline in various doses is therefore particularly useful in the long term treatment of nocturnal asthma, chronic pulmonary diseases having a broncho-obstructive component and all forms of broncho-obstructive reaction secondary to bronchial inflammatory diseases, including those secondary to cardiovascular diseases (the so-called chronic pulmonary heart), and in multiple therapy using drugs active on the cardiovascular system

The scope of the invention comprises all transdermal systems for the administration of doxofylline, which make it possible to achieve serum levels of between 0.1 and 300 mg/ml in treated individuals.

The transdermal forms of administration to which the invention relates may be provided in the form of a transdermal patch, for example having two layers (monolithic patch), three layers or in the form of a reservoir.

The formulation of a three-layer transdermal patch comprises:
a supporting barrier layer (backing) of polymer material, which is flexible and generally non-occlusive, which protects the underlying layer from external agents; this barrier layer may be constructed using polymer materials such as polyurethanes or polyamides or non-woven fabric (artificial silk);
a second layer of polymer matrix containing doxofylline;
a third layer of pressure-sensitive adhesive (PSA) containing, for example, polyvinylpyrrolidone or acrylate or polyisobutene, which may be protected by a silicone film, which protects it during storage and is removed prior to use.

The monolithic patch, which currently constitutes the preferred form of implementation, comprises a first backing layer of the type previously described and a second layer which imparts both adhesion and controlled release to the system. The polymer matrix material in which the active ingredient, doxofylline, is dispersed in the monolithic patch form of implementation, or three-layer patch, typically comprises polymer materials such as acrylates, silicones, block copolymers, including in particular polystyrene derivatives; examples of styrene block copolymers having adhesive properties which can be used for monolithic patches comprise, for example, styrene-isoprene-styrene (SIS), styrene-butadiene-styrene (SBS) and styrene-ethylene/butylene-styrene (SEBS) block copolymers.

In a reservoir formulation, which is in itself known, doxofylline is contained in a solution/gel in contact with a membrane which controls its release to the skin.

In one embodiment the high permeability of doxofylline makes it possible to formulate transdermal administration systems which do not include chemical agents potentiating epithelial penetration, that is those agents which increase permeability by fluidising the lipid structure of the corneal layer, such as for example, azone, DMSO, alcohols, fatty acids, terpenes, terpenoids, essential oils and their mixtures. However it is intended that transdermal administration systems comprising one or more potentiating agents such as those mentioned above and their mixtures are included within the scope of the invention.

Tests relating to determination of the permeability of doxofylline in comparison with reference active agents have been carried out using the procedures below.

### 1. Permeability Tests

1.1 Comparison of Doxofylline with Standard Drugs

MDCK cells were kept in Minimal Essential Medium Eagle (EMEM) containing 10% FBS, 2 nM of fresh L-glutamine and 2 nM of penicillin-streptomycin. The culture inserts were inseminated with 664000 cells per cm² (0.33 cm² per insert). The monolayers of MDCK were cultured for three days before use. The monolayers were washed with Hanks Balanced Salt Solution (HBSS+10 nM Hepes pH 7.4) transport medium; the electrical resistance of each monolayer was measured at 37° C. using a Millicell ERS-2 (Millipore). Background resistance values were subtracted from the resistance values of treated wells of monolayer. The resulting value was multiplied by the area of the membrane (0.33 cm²) to calculate the transepithelial electrical resistance values (TEER (Ωcm²)) for each monolayer. TEER values indicate the degree of confluence of the monolayer and the development of tight junctions. If the resistance was <200 Ωcm² the test was continued. MDCK monolayers having TEER values >200 Ωcm² were not used. The donor solutions in the transport test comprised 100 μM of test compound in the transport medium containing 100 nM of fluorescein isocyanate-dextran. The transport tests were performed using 0.250 ml of apical donor solution and 1 ml of basolateral acceptor solution (transport medium, pH 7.4). The internal standards and doxofylline were tested using five replicates. The monolayers were incubated with donor and acceptor solutions for 120 minutes at 37° C. The basolateral compartments were sampled after 120 minutes and analysed using LC-MS/MS, together with an aliquot of the donor solutions in the transport test (100 μM of the test compound) corresponding to t₀ (sample at time zero). In the quality check the internal standards (atenolol, dexamethasone, tartrate salt with metoprolol and fluorescein isocyanate-dextran) were tested under the same conditions as the test compound. Pₐₚₚ (apparent permeability) values were calculated in accordance with the following equation:

$${P}_{A - B} = \frac{\frac{d Q}{d t}}{A \cdot {C}_{A}} = \frac{\frac{Δ Q}{Δ t}}{A \cdot {C}_{A}}$$

in which dQ/dt is the permeation velocity, C_{A} is the initial concentration in the donor compartment and A is the surface area of the filter. Permeation velocities were calculated by preparing a diagram of the percentage of mass of active ingredient drug (peak area) found in the basolateral compartment as a function of time.

The results for fluorescein isothiocyanate/dextran were used as an internal control for each monolayer, to check the integrity of the tight junction throughout the period of the test. The normal range for fluorescein isocyanate-dextran permeability in MDCK monolayers is approximately 1 to 7 nM/second. The results from MDCK monolayers with fluorescein isothiocyanate/dextran Pₐₚₚ<10 nM/S were not used in calculating the Pₐₚₚ for the compound.

LC-MS/MS Analyses

The following HPLC conditions were used for all the tests:
column: Kinetex C₈-2.6 μM, 2.1×50 mm and Phenomenex
solvent A: 1 mM NH₄ AC
solvent B: 100% CH₃ CN.

The gradient used in HPLC separation was:

The T₀ samples (100 μM) were diluted in 50:50 solvent A: solvent B before analysis.

The dilution factors used were:
atenolol 1:400
metoprolol 1:200
dexamethasone 1:40
doxofylline 1:26.

The integrated peak area for the LC-MS/MS chromatograms was used as the instrument response (INRE) for relative quantification of the test material.

The permeability of the test material was then evaluated by comparing the INRE for five replicates after two hours incubation, on the basis of the INRE response to the t₀ samples. The results obtained after incubation with fluorescein isocyanate-dextran demonstrate that the monolayer remained intact (see table 1). The results obtained from incubation with the internal standards and doxofylline show that doxofylline has a high permeability value (table 2).

TABLE 1
Transport well treated with atenolol, metoprolol, dexamethasone and
doxofylline for 2 hours; fluorescein isothiocyanate-dextran was read
using fluorescence at 520 nM-490 nM TECAN INFINIT M200 Plate
reader to check the integrity of the monolayer.

| | ABS (mean of 5 | | |
|---|---|---|---|
| | transport wells) | nanoMolare | Pₐₚₚ (nM/sec) |
| atenolol | 36.4 | 0.10 | 4.2 |
| metoprolol | 34 | 0.10 | 4.2 |
| dexamethasone | 27 | 0.08 | 3.37 |
| doxofylline | 28 | 0.08 | 3.37 |

TABLE 2
Pₐₚₚ for atenolol, metoprolol, dexamethasone and doxofylline.

| Compounds | Pₐₚₚ (nM/sec) |
|---|---|
| Atenolol (low permeability standard) | 6.82 |
| Metoprolol (high permeability standard) | 65.15 |
| Dexamethasone (medium permeability standard) | 46.63 |
| Doxofylline | 226.64 |

Through the MTT test using human keratinocyte cell lines (HACAT), human fibroblast cell lines (HFF) and human endothelial cells from the umbilical vein (HUVEC) it was also demonstrated that doxofylline has no cytotoxicity.

### 1.2 Comparison Between Doxofylline and Theophylline

The skin permeability of doxofylline and theophylline was compared using an in vitro-ex vivo experimental protocol across human epidermis using Franz diffusion cells.

The Franz diffusion cells comprise two compartments, one containing the active ingredient (donor vehicle) and the other containing a receiving solution, separated by a slice of human epidermis. The vertical cells used in the actual experimental unit have a wider column than that in the original Franz type of diffusion cell, and the decanter shape was dispensed with. These have a diffusion area of 0.636 cm² and a receiving compartment of approximately 3.0 ml. The receiving volume in each cell is individually calibrated and equipped with a magnetic stirrer.

### Materials:

Theophylline WS RSNC 004 A, batch No. TB-120076
Doxofylline WS, batch No. 15530613

### In Vitro Penetration Test

Human epidermis was selected as the membrane for skin permeation studies. The skin used in the studies was obtained from the abdominal skin of a donor who underwent cosmetic surgery. The skin samples were prepared following an internal standard procedure. The full thickness of the skin was sealed in evacuated plastic bags and frozen at −20° C. within 6 hours of removal. Before the experiments the skin was thawed out at ambient temperature and excess fat was completely removed. The sections of skin were cut into squares of approximately 2.5 cm² and after the skin had been immersed in water at 60° C. for one minute the epidermis was separated off from the remaining tissue using forceps. The integrity of the skin was evaluated by measuring the impedance of each sample (Agilent LCR Meter 4263B).

Subsequently the epidermis was mounted in the Franz diffusion cell, the receiving compartment of which was filled with saline solution (NaCl 0.9% w/v) filtered using a 0.22 μm filter (VWR, nylon membrane). Particular attention was paid to avoiding air bubbles between the cover and the epidermis in the receiving compartment. The top and bottom parts of the Franz cell were sealed with Teflon and Parafilm® (Pechiney Plastic Packaging Company, Chicago, USA) and attached together by means of a clamp with the epidermis acting as a seal between the donor and receiving compartments. The donor compartment was then filled with 0.5 ml of a solution of theophylline in water or in triacetin (4% w/v). A precipitate was observed in these solutions, and these were therefore filtered using a 0.45 μm filter (VWR, polypropylene membrane) before being placed in the cells. A portion of each was preserved and diluted 1:2 in water or triacetin.

The system was maintained at 37° C. using a circulating water bath, so that the temperature of the surface of the epidermis was 32±1° C. during the test. At predetermined times (1, 3, 6, 24 hours) samples of 0.2 ml were taken from the receiving compartment and replaced with fresh receiving medium. “Sink” conditions were maintained during the tests.

The experimental conditions are summarised in the table below:

| | |
|---|---|
| Method | Modified Franz diffusion systems |
| Volume of the receiving | ≈3 ml |
| compartment | |
| Dimensions of the test sample | 0.636 cm² |
| Medium | Saline solution |
| Temperature | 32 ± 1° C. |
| Volume of sample removed | 0.2 ml |
| Sampling times | 1 h, 3 h, 6 h, 24 h |

The tests performed using theophylline were repeated in the same way using a solution of doxofylline in water or triacetin (4% w/v).

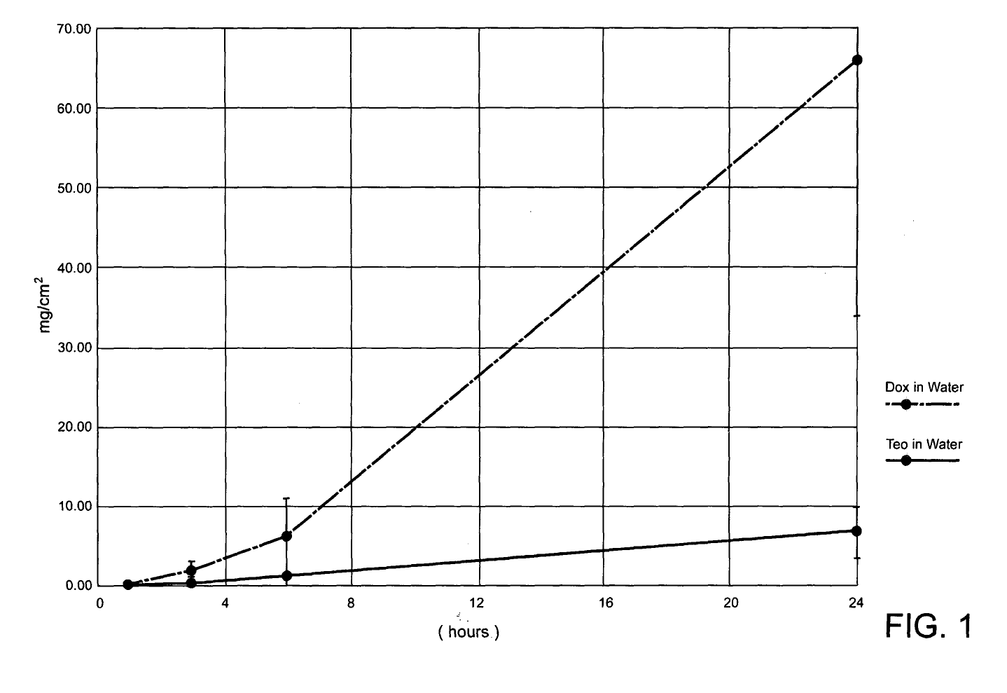

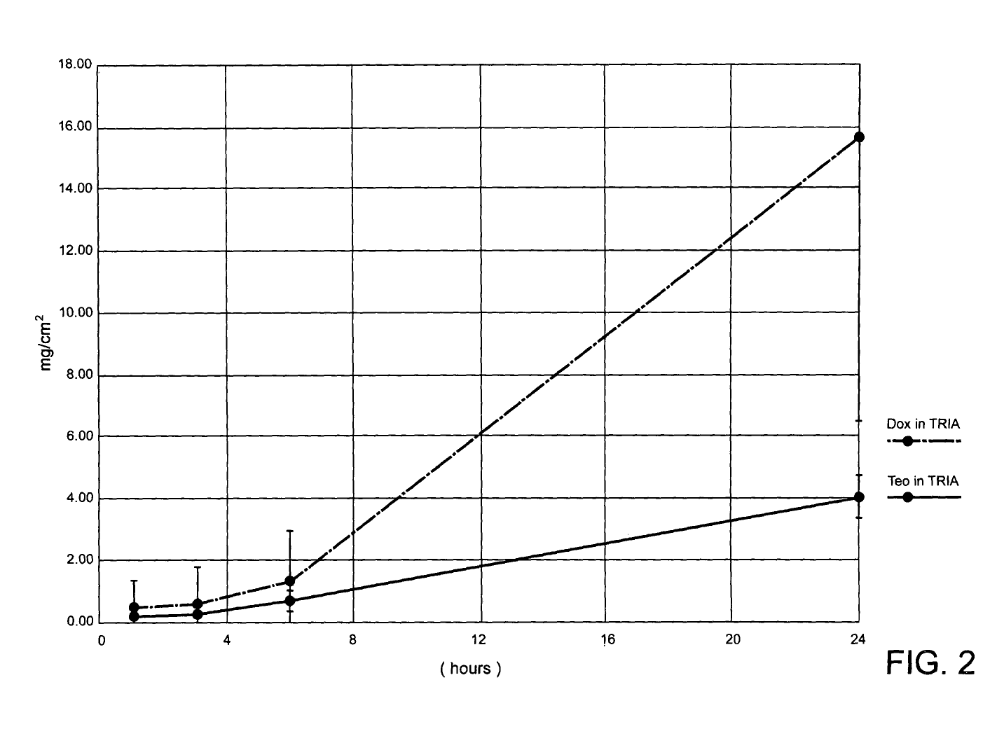

The results obtained are shown in the graphs in FIGS. 1 and 2.

The graph in FIG. 1 shows the values of doxofylline and theophylline permeate expressed as micrograms/cm² of surface area over time (4 sample times up to 24 hours) with aqueous solvent.

The graph in FIG. 2 similarly shows the values for doxofylline and theophylline permeate in triacetin solvent.

The results clearly demonstrate that the skin permeability values for doxofylline are substantially higher than those for theophylline in both the solvents used.

1. System for transdermal administration comprising doxofylline as the active ingredient for the use in the treatment of bronchopulmonary diseases having a broncho-obstructive component in an adult patient.

2. System for administration according to claim 1, for use in the long term treatment of asthma, nocturnal asthma, chronic lung diseases having a broncho-obstructive component, forms of broncho-obstructive reaction secondary to bronchial inflammatory diseases or cardiovascular diseases, optionally in multiple treatment with drugs active on the cardiovascular system.

3. System for administration according to claim 1, in the form of a reservoir.

4. System for administration according to claim 1, in the form of a transdermal patch comprising a first supporting polymer barrier layer, a second layer of polymer matrix containing doxofylline and a third layer comprising a pressure-sensitive adhesive.

5. System for administration according to claim 1, in the form of a transdermal patch comprising a supporting layer of polymer material and a second polymer layer of matrix including doxofylline, having adhesive properties.

6. System for administration according to claim 4, in which the matrix polymer is selected from the group comprising acrylic and silicone (co)polymers and styrene block copolymers.

7. System for administration according to claim 1, without any agents potentiating permeation of the active ingredient.

8. Doxofylline for use in the therapeutic treatment of chronic bronchopulmonary diseases, having a broncho-obstructive component, in an adult patient by transdermal administration.

9. Doxofylline according to claim 8, for use in the therapeutic treatment of asthma, nocturnal asthma, forms of broncho-obstructive reaction secondary to bronchial inflammatory diseases and secondary to cardiovascular diseases.
